(19) United States
(12) Patent Application Publication
(10) Pub. No.: US 2025/0094484 A1
(43) Pub. Date: Mar. 20, 2025

(54) SYSTEMS AND METHODS FOR LANGUAGE-GUIDED IMAGE RETRIEVAL

(71) Applicant: Shanghai United Imaging Intelligence Co., Ltd., Shanghai (CN)

(72) Inventors: Meng Zheng, Cambridge, MA (US); Ziyan Wu, Lexington, MA (US); Benjamin Planche, Brianwood, NY (US); Zhongpai Gao, Rowley, MA (US); Terrence Chen, Lexington, MA (US)

(73) Assignee: Shanghai United Imaging Intelligence Co., Ltd., Shanghai (CN)

(21) Appl. No.: 18/369,766

(22) Filed: Sep. 18, 2023

(51) Int. Cl. G06F 16/583; G06F 16/58; G06V 10/44; G06V 10/82; G06V 20/70; G16H 30/40

(52) U.S. Cl. CPC G06F 16/583; G06F 16/5866; G06V 10/44; G06V 10/82; G06V 20/70; G16H 30/40

(57) ABSTRACT

Described herein are machine learning (ML) based on systems, methods, and instrumentalities associated with image search and/or retrieval. An apparatus as described herein may obtain a query image and a textual description associated with the query image, and generate, using an artificial neural network (ANN), a feature representation that may represent the image and the textual description as an associated pair. Based on the feature representation, the apparatus may identify one or more images from an image repository and provide an indication regarding the one or more identified images, for example, as a ranked list.

### BACKGROUND

Image search and retrieval have become increasingly popular with the rise of computer vision and video based social media. Conventional image retrieval techniques focus on matching image properties and are incapable of capturing and considering the purpose of a search or the intent of a user, much less understanding the meaning of the purpose or intent when it is expressed in a natural language form, and using the understanding to improve the accuracy of image identification. Accordingly, systems and methods that are capable of overcoming the aforementioned shortcomings of the conventional image retrieval techniques are desirable.

### SUMMARY

Described herein are machine learning (ML) based on systems, methods, and instrumentalities associated with image search and retrieval. An apparatus as described herein may include one or more processors configured to obtain an image of a person and a textual description associated with the image, and generate a feature representation (e.g., one or more embeddings) that may represent the image of the person and the textual description as an associated pair. The feature representation may be generated using an artificial neural network (ANN), based on which the apparatus may be further configured to identify one or more images from an image repository and provide an indication regarding the one or more identified images, for example, as a ranked list (e.g., according to the respective relevance of the one or more images to the image of the person and the textual description).

In examples, the ANN described herein may include at least a first neural network, a second neural network, and a cross-attention module. The first neural network may be configured to extract features from the textual description associated with the image of the person, the second neural network may be configured to extract features from the image of the person, and the cross-attention module may be configured to establish a relationship between the features extracted from the image and the features extracted from the textual description.

In examples, at least one of the first neural network or the second neural network includes a transformer neural network, the features extracted from the textual description may be provided to the cross-attention module in one or more key matrices and one or more value matrices, and the features extracted from the image of the person may be provided to the cross-attention module in one or more query matrices. In examples, the second neural network may be configured to implement a machine-learning (ML) language model (e.g., a large language model (LLM)) that may be pre-trained to extract the features from the textual description and generate an embedding that represents the extracted features.

In examples, the feature representation that represents the image of the person and the textual description as an associated pair may be generated by conditioning the features extracted from the image of the person on the features extracted from the textual description, or by combining the features extracted from the image of the person with the features extracted from the textual description. In examples, the one or more images from the image repository may be tagged with respective textual descriptions, and the one or more images may be identified from the image repository further based on the textual descriptions used to tag the one or more images.

In examples, the image of the person described herein may include a medical scan image that may depict an anatomical structure of the person, the textual description associated with the image of the person may indicate an abnormality of the anatomical structure, and at least one of the one or more images identified from the image repository may depict the anatomical structure of a different person with a substantially similar abnormality. In examples, the textual description associated with the image of the person may differ, on a verbatim basis, from at least one of the textual descriptions used to tag the one or more images.

### BRIEF DESCRIPTION OF THE DRAWINGS

A more detailed understanding of the examples disclosed herein may be had from the following description, given by way of example in conjunction with the accompanying drawing.

FIG. 1 is a simplified block diagram illustrating an example of language-guided image retrieval using ML-based techniques.

FIG. 2 is a simplified block diagram illustrating an example of an artificial neural network that may be used to perform an image search and/or retrieval task based on a query image and a description associated with the query message.

FIG. 3 is a simplified block diagram illustrating example operations that may be associated with training an artificial neural network to perform the image search and/or retrieval tasks described herein.

FIG. 4 is a flow diagram illustrating example operations that may be associated with training a neural network to perform one or more of the tasks described herein.

FIG. 5 is a block diagram illustrating example components of an apparatus that may be configured to perform the tasks described herein.

### DETAILED DESCRIPTION

The present disclosure is illustrated by way of example, and not by way of limitation, in the figures of the accompanying drawings. A detailed description of illustrative embodiments will be provided with reference to the figures. Although these embodiments may be described with certain technical details, it should be noted that the details are not intended to limit the scope of the disclosure. Further, while some embodiments may be described in a medical setting, those skilled in the art will understand that the techniques disclosed in those embodiments may also be applicable to other settings or use cases.

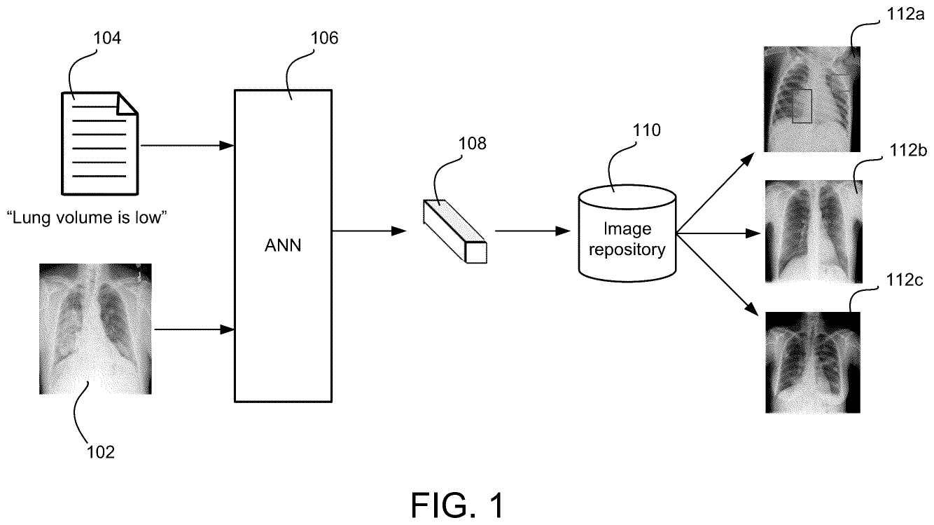

FIG. 1 illustrates an example of language-guided image search and/or retrieval. As shown, a computer-based system or apparatus (e.g., referred to herein “an image finder”) may be tasked with identifying and/or retrieving images that may resemble an input image 102 (e.g., also referred to herein as “a query image”) and match a description 104 (e.g., a textual description) of the input image 102. The image finder may be configured to obtain (e.g., receive or otherwise acquire) the query image 102 and the description 104 in various ways. For example, the query image 102 may be a color image, a depth image, a radar image, or an infrared (IR) image of a person, and the image finder may be configured to obtain the query image from a corresponding sensing device such as a red-green-blue (RGB) sensor, a depth sensor, a radar sensor, or an IR sensor that may be configured to capture the query image 102 of the person (e.g., from a location inside a medical facilitate). As another example, the query image 102 may be a medical scan image of a patient such as an X-ray image, a computed tomography (CT) image, or a magnetic resonance imaging (MRI) image of the patient, and the image finder may be configured to obtain the medical scan image from a medical image repository (e.g., database) or from a corresponding medical scanner such as an X-ray scanner, a CT scanner, or an MRI scanner.

In some examples, the description 104 may describe the query image 102 itself, while in other examples, the description 104 may described the purpose or intent of the search and/or retrieval task. For instance, when the query image 102 is a medical scan image that depicts an anatomical structure (e.g., lungs) of a patient, the description 104 may indicate an abnormality detected (e.g., by a physician) in the query image, such as, e.g., “lung volume is low.” As another example, when the description 104 indicates a purpose or intent of the search and/or retrieval task, the purpose or intent may be expressed as an instruction or command such as, e.g., “find scan images with similar lung volumes.” While the description 104 may be treated as a textual description in the examples provides herein, those skilled in the art will understand that the description 104 may also be in other formats such as an audio format without affecting the applicability of the techniques described herein (e.g., an audio description may be transcribed into texts).

The image finder may apply machine-learning (ML) techniques to accomplish the image search and/or retrieval task described herein. For example, the image finder may be configured to analyze the query image 102 and the description 104 using an artificial neural network (ANN) 106, and generate a feature representation 108 that may uniquely identify the query image 102 and description 104 as an associated pair. As will be described in greater detail below, the ANN 106 may include multiple neural networks (e.g., which may also be referred to herein as sub-neural networks or neural network modules) that may be configured to extract features from the query image 102 and the description 104, establish a relationship between the image features extracted from the query image 102 and the text features extracted from the description 104, and represent the extracted features and/or the correspondence between the image 102 and the description 104 via feature representation 108. The features and/or feature representations described herein may also be referred to as embeddings, which may take the form of a numerical vector in some examples.

Upon generating the feature representation 108, the image finder may use it to conduct a guided search in an image repository 110 to identify images (e.g., one or more images) that may match (e.g., resemble or be related to) the query image 102 and the description 104. In examples, the image repository 110 may include candidate images that are tagged with respective descriptions (e.g., textual descriptions such as natural language descriptions), and the image finder may identify one or more matching images (e.g., 112a, 112b and 112c shown in FIG. 1) from the repository by comparing the feature representation 108 (e.g., representing the query image 102 and the description 104 as an associated pair) with the feature representations (e.g., embeddings) of the candidate images and their corresponding descriptions (e.g., the embeddings associated with the candidate images may be determined according to the description 104 provided by a user). For example, when the query image 102 is a medical scan image of an anatomical structure and the description 104 indicates an abnormality of the anatomical structure (e.g., lung volume is low), the image finder may identify the one or more images 112a-112c based on a determination that those images have similar features as the query image 102, and are tagged with descriptions (e.g., diagnoses or observations) that indicate a substantially similar abnormality. As another example, when the query image 102 is a medical scan image of an anatomical structure (e.g., lungs) and the description 104 indicates that the purpose or intention of the search is to “find scan images with similar abnormalities located at the bottom of the left lung,” the image finder may identify the one or more images 112a-112c based on a determination that those images have similar features as the query image 102, and are tagged with descriptions (e.g., diagnoses, observations, and/or measurement results) that indicate that the images include abnormalities at the bottom of the left lung.

As a result of the search and/or identification operations, the image finder may provide an indication (e.g., as an output) regarding the one or more images 112a-112c identified from the image repository 110 via the matching process described above. The image finder may, for example, provide a ranking of the one or more images 112a-112c based on their respective relevance to the query image 102 and the description 104 (e.g., the image finder may calculate a respective matching score for each of the images 112a-112c). As another example, the image finder may retrieve the images 112a-112c and display them to a user (e.g., a physician) via a monitor or a virtual reality (VR)/augmented relative (AR) device. The image finder may also transmit the images 112a-112c to another device or apparatus, which may use the images for a downstream task.

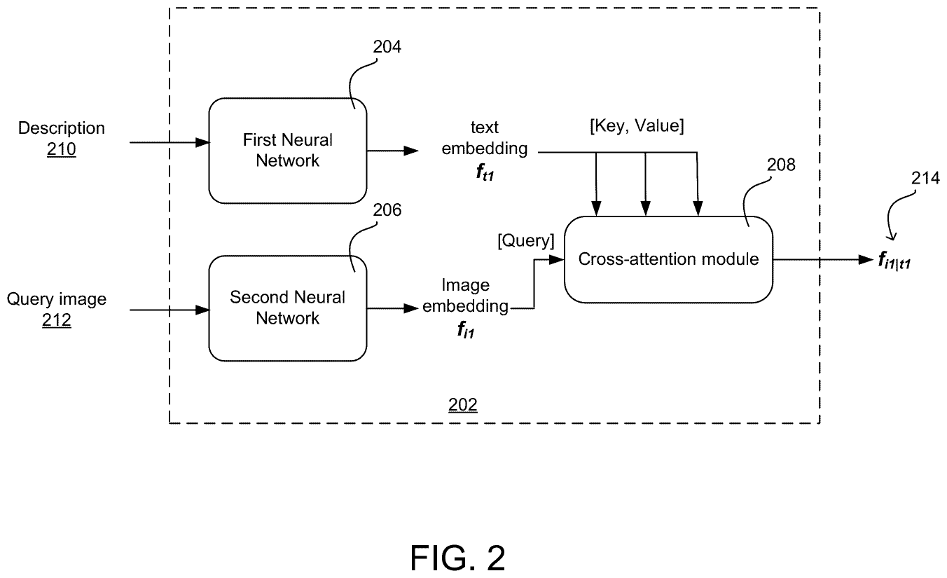

FIG. 2 illustrates an example architecture of an artificial neural network (ANN) 202 (e.g., ANN 106 of FIG. 1) that may be used to perform the tasks described herein. As shown, ANN 202 may include at least a first neural network 204, a second neural network 206, and a cross-attention module 208. The first neural network 204 may be configured to extract features from a description 210 (e.g., a textual description) associated with a query image 212 of a person, the second neural network 206 may be configured to extract features from the query image 212, and the cross-attention module 208 may be configured to determine (e.g., establish) a relationship between the respective features extracted from description 210 and query image 212, and generate a feature representation 214 that may represent the description 210 and the query image 212 as an associated pair.

The first neural network 204 may be configured to implement natural language processing (NLP) model such as a large language model (LLM) pre-trained for extracting features from the description 210 and generate an embedding that represents the extracted features. The NLP model may be capable of processing textual inputs of different formats including, for example, a word-level input, a sentence-level input, and/or a character-level input. With a word-level input, a plurality of tokens or word embeddings may be derived using a tokenizer (e.g., such as byte pair encoding (BPE) or sentencepiece), where each token or word embedding may represent the meaning of a word or sub-word. With a sentence-level input, an entire sentence may be treated as a single token, and the input sequence may include a sequence of sentence embeddings, making the input useful at least for tasks such as sentiment analysis and/or text classification (e.g., where the NLP model may be used to make predictions based on the overall meaning of the input text). With a character-level input, each token in the input text may correspond to a single character, and the input sequence may include a sequence of character embeddings, making the input useful at least for tasks such as named entity recognition (e.g., where the NLP model may be used to recognize rare or unseen words).

The first neural network 204 may be implemented using a transformer architecture. Such an architecture include an encoder and/or a decoder, each of which may include multiple layers. The encoder may be configured to receive a sequence of input tokens (e.g., such as words in a sentence) and generates a sequence of hidden representations (also referred to as embeddings) that may capture the meaning of each token. An encoder layer may include multiple (e.g., two) sub-layers, such as, e.g., a multi-head self-attention layer and a position-wise feedforward layer. The self-attention layer may allow the neural network to attend to different parts of the input sequence and learn relationships between them. For example, via the self-attention layer, a weighted sum of the input tokens may be calculated, where the relevant weights may be determined through a learned attention function that accounts for the similarity between each token and all other tokens in the sequence. The feedforward layer may then apply a non-linear transformation to each token's hidden representation, allowing the neural network to capture complex patterns in the input sequence. Residual connections and/or layer normalization may be used and/or applied after each sub-layer to stabilize the training process.

The decoder of the transformer architecture may be configured to receive a sequence of target tokens and generate a sequence of hidden representations (e.g., embeddings) that may capture the meaning of each target token, conditioned on the encoder's output. A decoder layer may also include multiple (e.g., two) sub-layers, such as, e.g., a masked multi-head self-attention layer, which may attend to target tokens that have already been generated, and a multi-head attention layer, which may attend to the encoder's output. The masked self-attention layer may allow the neural network to generate the target tokens one at a time, while preventing it from looking ahead in the sequence. The multi-head attention layer may attend to the encoder's output to help the neural network generate target tokens that may be semantically related to the input sequence. The decoder may also include a position-wise feedforward layer, and may use or apply residual connections and/or layer normalization after each sub-layer (e.g., similar to the encoder).

The second neural network 206 may also be implemented using a transformer architecture such as a vision transformer (ViT) architecture. The ViT architecture may include multiple (e.g., two) components, such as, e.g., a feature extractor and a transformer encoder. The feature extractor may include a convolutional neural network (CNN) configured to extract features (e.g., local features) from the query image 212. The extracted features may be flattened into a sequence of feature representations (e.g., such as two-dimensional (2D) feature vectors), which may be fed into the transformer encoder. The transformer encoder may include multiple layers, each of which may include a multi-head self-attention layer and/or a feedforward layer. The self-attention layer may allow the neural network to attend to different parts of the feature sequence and learn relationships between them, while the feedforward layer may apply a non-linear transformation to a (e.g., each) feature vector. Residual connections and layer normalization may be applied after a (e.g., each) sub-layer, for example, to stabilize the training process. Using such an architecture, an entire image may be processed at once, for example, without spatial pooling. This may be achieved by splitting the image into non-overlapping patches and treating patches as a sequence of feature vector input to the transformer encoder. The neural network may also include an additional learnable position embedding (e.g., for each patch), which may encode the respective spatial locations of the patches within the image. The output of the vision transformer may include a sequence of feature vectors, each of which may correspond to a different patch in the query image 212. These feature vectors may then be used for various downstream tasks, such as image classification or object detection, for example, by adding additional layers (e.g., task-specific programming logics) on top of the output sequence.

The respective feature representations (e.g., text embeddings fₜ₁ and image embedding fᵢ₁) generated by the first neural network 204 and the second neural network 206 may be linked by the cross-attention module 208, for example, via feature representation 214. The linking may be accomplished, for example, by exchanging the key, value, and/or query matrices of the first neural network 204 and the second neural network 206 that may be derived as parts of the transformer architecture. For instance, as illustrated by FIG. 2, the text embedding fₜ₁ generated by first neural network 204 may be provided to the cross-attention module 208 in one or more key matrices and one or more value matrices, while the image embedding fᵢ₁ may be provided to the cross-attention module 208 in one or more query matrices. As a result, the feature representation 214 generated by the cross-attention module 208 may correspond to a conditioning of the image embedding fᵢ₁ on the text embedding fₜ₁, denoted as f_{i1|t1} (e.g., the cross-attention module 208 may combine asymmetrically the image and text embeddings of a same dimension for information fusion across two different modalities). In examples, the feature representation 214 may also correspond to a simple combination (e.g., concatenation) of the image embedding fᵢ₁ and the text embedding fₜ₁.

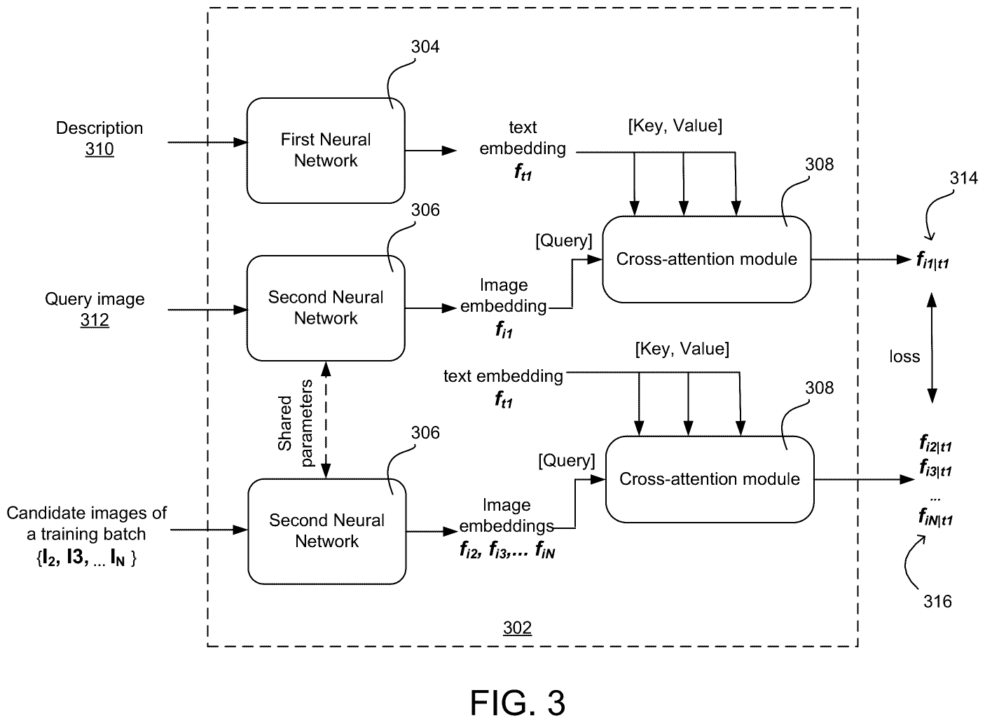

FIG. 3 illustrates example operations that may be associated with training an artificial neural network 302 (e.g., ANN 202 of FIG. 2) to perform the tasks described herein. As shown, the artificial neural network 302 may include a first neural network 304, a second neural network 306, and a cross-attention module 308, corresponding to first neural network 204, second neural network 206, and cross-attention module 208 of FIG. 2, respectively. Also as shown, the training of the artificial neural network 302 may be conducted based on a description 310 associated with a query image 312, the query image 312, and a set of candidate images [I₂, I₃, . . . . I_{N}]. During the training, the first neural network 304, the second neural network 306, and the cross-attention module 308 may be used to generate a feature representation 314 (e.g., f_{i1|t1}) using the techniques described with reference to FIG. 2, wherein the feature representation 314 may represent the description 310 and the query image 312 as an associated pair. Similar to the query image 312, candidate images [I₂, I₃, . . . . I_{N}] may also be processed via the second neural network 306 to derive image embeddings [fᵢ₂, fᵢ₃, . . . f_{iN}] (e.g., two instances of the second neural network 306 sharing a same set of parameters may be used during the training to process the query image 312 and each candidate image, respectively). The candidate image embeddings may then be linked to the text embedding fₜ₁ (e.g., using the cross-attention module 308) to derive candidate feature representations 316 (f_{i2|t1}, f_{i3|t1}, . . . f_{iN|t1}), which may then be compared to feature representation 314 (e.g., f_{i1|t1}) to determine a loss for adjusting the parameters of the neural networks involved.

In examples, the training process described above may be performed in a self-supervised or weakly supervised manner (e.g., by utilizing text information paired with original training images). For instance, the training may be performed using a contrastive learning technique, with which a contrastive loss function (e.g., a distance-based contrastive loss function or a cosine similarity based contrastive loss function) may be used to calculate a difference between feature representation 314 corresponding to the query image 312 and feature representation 316 corresponding to the candidate image I_{N}. The parameters of ANN 302 may then be adjusted with an objective to minimize the difference when the candidate image I_{N} is similar to the query image 312, and to maximize the difference when the candidate image I_{N} is dissimilar to the query image 312. As shown in FIG. 3, such an objective may be accomplished by arranging one or more of the neural networks in a twin (e.g., Siamese) structure (e.g., as shown by the parameter-sharing instances of second neural network 306), where one of the twin networks may be configured to process the query image 312 and the other of the twin networks may be configured to process the candidate image I_{N}. The respective feature representations (e.g., embeddings) produced by the twin networks may then be fed to the contrastive loss function to calculate a loss, and the gradient descent of the loss may be backpropagated through the neural network to adjust its parameters.

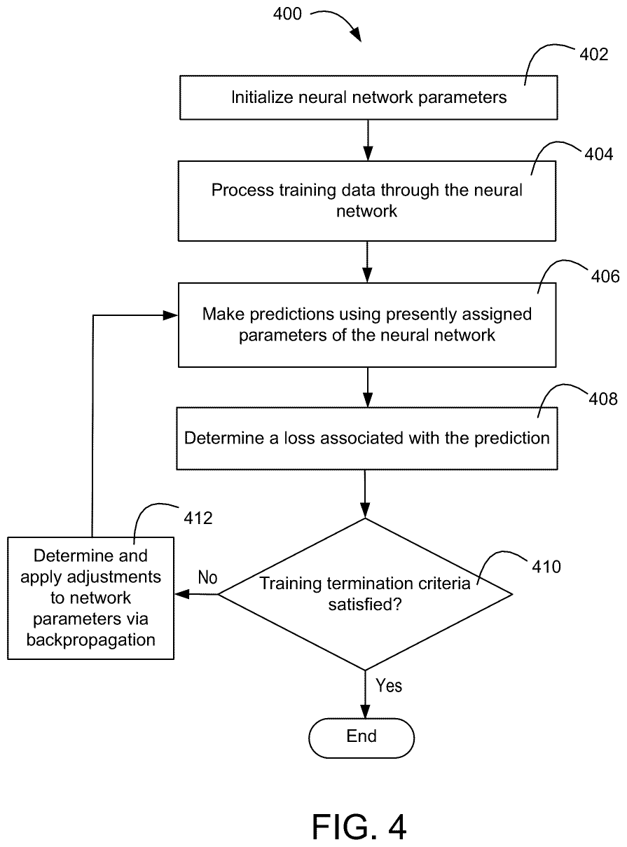

FIG. 4 illustrates example operations 400 that may be associated with training an artificial neural network (e.g., ANN 106 of FIG. 1 or ANN 202 of FIG. 2) to perform one or more of the tasks described herein. As shown, the training operations 400 may include initializing the operating parameters of the neural network (e.g., weights associated with various layers of the neural network) at 402, for example, by sampling from a probability distribution or by copying the parameters of another neural network having a similar structure. The training operations 400 may further include processing one or more first inputs (e.g., a query image and/or a description associated with the query image) using presently assigned parameters of the neural network at 404, and making a prediction for a first result (e.g., a first feature representation) at 406. The training operations 400 may further include processing one or more second inputs (e.g., a candidate image and/or a description associated with the candidate image) using presently assigned parameters of the neural network at 404, and making a prediction for a second result (e.g., a second feature representation) at 406. The predictions may then be used to calculate a loss at 408, for example, using a distance-based or cosine similarity based contrastive loss function.

At 410, the loss calculated at 408 may be used to determine whether one or more training termination criteria are satisfied. For example, the training termination criteria may be determined to be satisfied if the loss between predictions made on similar inputs is smaller than a threshold and/or if the loss between predictions made on dissimilar inputs is larger than a threshold. If the determination at 410 is that the termination criteria are satisfied, the training may end; otherwise, the presently assigned network parameters may be adjusted at 412, for example, by backpropagating a gradient descent of the loss through the neural network, before the training returns to 406.

For simplicity of explanation, the training operations 400 are depicted and described with a specific order. It should be appreciated, however, that the training operations 400 may occur in various orders, concurrently, and/or with other operations not presented or described herein. Furthermore, it should be noted that not all operations that may be included in the training method are depicted and described herein, and not all illustrated operations are required to be performed.

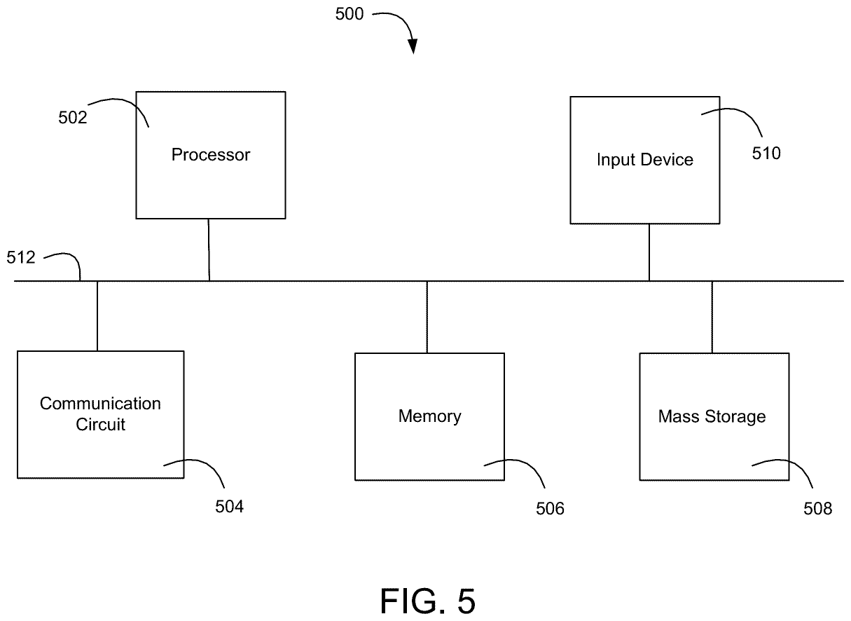

The systems, methods, and/or instrumentalities described herein may be implemented using one or more processors, one or more storage devices, and/or other suitable accessory devices such as display devices, communication devices, input/output devices, etc. FIG. 5 illustrates an example apparatus 500 that may be configured to perform the automatic image annotation tasks described herein. As shown, apparatus 500 may include a processor (e.g., one or more processors) 502, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 500 may further include a communication circuit 504, a memory 506, a mass storage device 508, an input device 510, and/or a communication link 512 (e.g., a communication bus) over which the one or more components shown in the figure may exchange information.

Communication circuit 504 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 506 may include a storage medium (e.g., a non-transitory storage medium) configured to store machine-readable instructions that, when executed, cause processor 502 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 508 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 502. Input device 510 may include a keyboard, a mouse, a voice-controlled input device, a touch sensitive input device (e.g., a touch screen), and/or the like for receiving user inputs to apparatus 500.

It should be noted that apparatus 500 may operate as a standalone device or may be connected (e.g., networked, or clustered) with other computation devices to perform the functions described herein. And even though only one instance of each component is shown in FIG. 5, a skilled person in the art will understand that apparatus 500 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. An apparatus, comprising:
one or more processors configured to:
obtain an image of a person;
obtain a textual description associated with the image;
generate, using an artificial neural network (ANN), a feature representation that represents the image of the person and the textual description as an associated pair;
identify one or more images from an image repository based on at least the feature representation generated using the ANN; and
provide an indication regarding the one or more images identified from the image repository.

2. The apparatus of claim 1, wherein the ANN includes at least a first neural network, a second neural network, and a cross-attention module, the first neural network configured to extract features from the textual description associated with the image of the person, the second neural network configured to extract features from the image of the person, the cross-attention module configured to establish a relationship between the features extracted from the image and the features extracted from the textual description.

3. The apparatus of claim 2, wherein at least one of the first neural network or the second neural network includes a transformer neural network.

4. The apparatus of claim 3, wherein the features extracted from the textual description are provided to the cross-attention module in one or more key matrices and one or more value matrices, and wherein the features extracted from the image of the person are provided to the cross-attention module in one or more query matrices.

5. The apparatus of claim 2, wherein the second neural network is configured to implement a machine-learning (ML) language model that is pre-trained to extract the features from the textual description and generate an embedding that represents the extracted features.

6. The apparatus of claim 1, wherein the feature representation is generated by conditioning the features extracted from the image of the person on the features extracted from the textual description, or by combining the features extracted from the image of the person with the features extracted from the textual description.

7. The apparatus of claim 1, wherein the one or more images from the image repository are tagged with respective textual descriptions, and wherein the one or more processors are configured to identify the one or more images further based on the respective textual descriptions used to tag the one or more images.

8. The apparatus of claim 7, wherein the textual description associated with the image of the person differs, on a verbatim basis, from at least one of the textual descriptions used to tag the one or more images.

9. The apparatus of claim 7, wherein the image of the person includes a medical scan image that depicts an anatomical structure of the person, wherein the textual description associated with the image of the person indicates an abnormality of the anatomical structure, and wherein at least one of the one or more images identified from the image repository depicts the anatomical structure of a different person with a substantially similar abnormality.

10. The apparatus of claim 1, wherein the one or more processors being configured to provide the indication regarding the one or more images identified from the image repository comprises the one or more processors being configured to provide a ranking of the one or more images based on respective relevance of the one or more images to the image of the person.

11. A method, comprising:
obtaining an image of a person;
obtaining a textual description associated with the image;
generating, using an artificial neural network (ANN), a feature representation that represents the image of the person and the textual description as an associated pair;
identifying one or more images from an image repository based on at least the feature representation that represents the image of the person and the textual description as the associated pair; and
providing an indication regarding the one or more images identified from the image repository.

12. The method of claim 11, wherein the ANN includes at least a first neural network, a second neural network, and a cross-attention module, the first neural network configured to extract features from the textual description associated with the image of the person, the second neural network configured to extract features from the image of the person, the cross-attention module configured to establish a relationship between the features extracted from the image and the features extracted from the textual description.

13. The method of claim 12, wherein at least one of the first neural network or the second neural network includes a transformer neural network.

14. The method of claim 13, wherein the features extracted from the textual description are provided to the cross-attention module in one or more key matrices and one or more value matrices, and wherein the features extracted from the image of the person are provided to the cross-attention module in one or more query matrices.

15. The method of claim 12, wherein the second neural network is configured to implement a machine-learning (ML) language model that is pre-trained to extract the features from the textual description and generate an embedding that represents the extracted features.

16. The method of claim 11, wherein the feature representation is generated by conditioning the features extracted from the image of the person on the features extracted from the textual description, or by combining the features extracted from the image of the person with the features extracted from the textual description.

17. The method of claim 11, wherein the one or more images from the image repository are tagged with respective textual descriptions, and wherein the one or more images are identified further based on the respective textual descriptions used to tag the one or more images.

18. The method of claim 17, wherein the image of the person includes a medical scan image that depicts an anatomical structure of the person, wherein the textual description associated with the image of the person indicates an abnormality of the anatomical structure, and wherein at least one of the one or more images identified from the image repository depicts the anatomical structure of a different person with a substantially similar abnormality.

19. The method of claim 11, wherein the textual description associated with the image of the person differs, on a verbatim basis, from at least one of the textual descriptions used to tag the one or more images.

20. A non-transitory computer-readable medium comprising instructions that, when executed by a processor included in a computing device, cause the processor to implement the method of claim 11.
